(19) United States
(12) Patent Application Publication
(10) Pub. No.: US 2007/0260236 A1
(43) Pub. Date: Nov. 8, 2007

(54) Radio frequency communication devices using chaotic signal and method thereof

(75) Inventors: Sang-Min Han, Hwaseong-si (KR); Young-hwan Kim, Hwaseong-si (KR); Mi-hyun Son, Seoul (KR); Jae-hyon Kim, Suwon-si (KR); Seong-soo Lee, Suwon-si (KR)

Correspondence Address: SUGHRUE MION, PLLC, 2100 PENNSYLVANIA AVENUE, N.W., SUITE 800, WASHINGTON, DC, 20037, US

(73) Assignee: SAMSUNG ELECTRONICS CO., LTD.

(21) Appl. No.: 11/512,377

(22) Filed: Aug. 30, 2006

(30) Foreign Application Priority Data
Apr. 14, 2006 (KR) 2006-34191

(51) Int. Cl. A61B 18/18

(52) U.S. Cl. 606/41

(57) ABSTRACT

RF (radio frequency) communication devices which use a chaotic signal and a method thereof are provided. An RF communication system includes: a transmitter terminal which generates a chaotic signal including a plurality of frequency components in a predetermined frequency band and transmits a chaotic carrier generated by inserting a data signal including information to be transmitted into the chaotic signal to a receiver terminal; and the receiver terminal extracts the data signal from the chaotic carrier received from the transmitter terminal via a wireless network and outputs the extracted data signal. As a result, low power, light weight local area RF communication devices can be realized.

### CROSS-REFERENCE TO RELATED APPLICATIONS

This application claims the benefit of Korean Patent Application No. 10-2006-34191 filed Apr. 14, 2006, in the Korean Intellectual Property Office, the disclosure of which is incorporated herein by reference.

### BACKGROUND OF THE INVENTION

1. Field of the Invention

The present invention relates to radio frequency (RF) communication devices using a chaotic signal and a method thereof. More particularly, the present invention relates to low power, light RF communication devices using a chaotic signal and a method thereof.

2. Description of the Related Art

Bluetooth-based RF headsets with which users are able to freely use their both hands during telephonic communications have been expanding in the market, and are becoming very popular. Bluetooth is a standard which allows digital devices such as computers and mobile terminals or electric home appliances positioned at short distances to be connected to each other wirelessly so as to enable real-time two-way communications among the digital devices.

In order to enable communications between a main device and a sub-device, i.e., between digital devices, for example, between a portable remote terminal and an RF headset, between a computer and an RF headset, or between a computer and a peripheral device, using such Bluetooth, the sub device as well as the main device must include an RF communication system having a transmitter and a receiver for RF communications.

Conventional RF communication systems use spread spectrum communication systems in which bandwidths of signals to be transmitted are spread to much wider bandwidths and then transmitted and sine waves, pulses, or the like are used as carriers. Carriers such as sine waves, pulses, or the like must be boosted to predetermined frequencies to transmit data. For this purpose, a transmitter requires elements for up-converting carriers to predetermined frequencies in a base band, and a receiver requires elements for down-converting carriers having boosted frequencies in a base band.

In more detail, a conventional RF communication system must include a voltage controlled oscillator (VCO) for generating a frequency necessary for a transmission of data and a phase-locked loop (PLL) for fixing the frequency generated by the VCO so that the frequency is not changed by an external effect. Also, a transmitter must include an up-mixer for up-converting carriers in a base band to the frequency generated by the VCO, and a receiver must include a down-mixer for down-converting the carriers to the based band.

However, if the conventional RF communication system includes the VCO, the PLL, the up-mixer, and the down-mixer, a large amount of power is consumed, and the great sizes of the up-mixer and the down-mixer increase the size of the conventional RF communication system.

Mobile sub-devices, for example, RF headsets, cannot be supplied with power using electric wires, and thus require chargers, batteries, or the like. However, transmitters and receivers consume large amounts of power as described above, and thus capacities of batteries are increased. Accordingly, the weight of the sub-devices is increased, and life-spans of the batteries are shortened.

A method of transmitting information using a chaotic signal has been recently suggested according to IEEE 802.15.4a standards.

IEEE 802.15.4a is a next generation communication field in which a positioning function and a low power function are added to mixed technology of 802.15.4 (ZigBee) and 802.15.3 (Ultra Wide Band: UWB) as Positioning Lower Power Detector Network Standard Groups.

In the present invention, a chaotic signal modulation method has been suggested to achieve the low power function. The chaotic signal modulation method does not require a VCO, a PLL, a mixer, and so on that can be designed in a simple RF structure in hardware and are required in such conventional RF communication systems as described above. Thus, if the chaotic signal modulation method is used, the amount of consumed power may be reduced to 10 mW, i.e., about ⅓ of the amount of consumed power in the prior art.

Accordingly, a chaotic signal modulation method is used to design an RF communication system so as to enable radio communications between a main device and a sub-device. As a result, a low power, compact RF communication system may be realized.

### SUMMARY OF THE INVENTION

Accordingly, the present general inventive concept has been made to address the above-mentioned problems, and an aspect of the present general inventive concept is to provide low power, compact RF communication devices using a chaotic signal and a method thereof.

According to an aspect of the present invention, there is provided an RF (radio frequency) communication system including: a transmitter terminal generating a chaotic signal comprising a plurality of frequency components in a predetermined frequency band and transmitting a chaotic carrier generated by inserting a data signal comprising information to be transmitted into the chaotic signal to a receiver terminal, the receiver terminal extracting the data signal from the chaotic carrier received from the transmitter terminal via a wireless network.

The transmitter terminal may include: a chaotic signal generator which generates the chaotic signal; a modulator which synthesizes the data signal with the chaotic signal to generate the chaotic carrier; and an antenna which transmits the chaotic carrier generated by the modulator.

The receiver terminal may include a detector which senses the chaotic carrier and extracts the data signal.

The transmitter terminal may further include a signal processing circuit which converts the data signal to a based band and outputs a digital signal. The signal processing circuit may be a mobile station modem of a portable remote terminal.

The transmitter terminal may further include an audio interface which receives an analog data signal from an external source and an ADC (analog-to-digital converter) which converts the analog data signal output from the audio interface into a digital data signal. The transmitter terminal may be a portable remote terminal, a computer, an MP3 (MPEG Audio Layer-3) player, an RF transceiver, a TV (television), an audio system, a PMP (portable multimedia player), a PDA (personal digital assistant), an RF headset, an RF mouse, or a computer peripheral device.

The receiver terminal may further include an ADC which converts the extracted digital data signal into an analog data signal. The receiver terminal may further include an audio interface which outputs the analog data signal. The receiver terminal may be a portable remote terminal, a computer, an MP3 player, an RF transceiver, a TV, an audio device, a PMP, a PDA, an RF headset, an RF mouse, a compute peripheral device, or a speaker.

According to another aspect of the present invention, there is provided an RF communication device which generates a chaotic signal including a plurality of frequency components in a predetermined frequency band and transmits a chaotic carrier generated by inserting a data signal including information to be transmitted into the chaotic signal to a receiver terminal.

According to another aspect of the present invention, there is provided an RF communication device which receives a chaotic carrier generated by inserting a data signal into a chaotic signal including a plurality of frequency components in a predetermined frequency band via a wireless network, extracting the data signal from the chaotic carrier, and processing the data signal.

According to another aspect of the present invention, there is provided an RF communication device including: a transmitter which generates a chaotic signal including a plurality of frequency components in a predetermined frequency band and transmits a chaotic carrier generated by inserting a data signal including information to be transmitted into the chaotic signal to a receiver; and the receiver extracts the data signal from the chaotic carrier received through an antenna and processes the extracted data signal.

The RF communication device may further include: a switch which connects the transmitter or the receiver to the antenna; and a BPF (band pass filter) which filters the chaotic carrier transmitted or received through the antenna. The RF communication device may further include a duplexer which connects the transmitter or the receiver to the antenna and filters the chaotic carrier transmitted or received through the antenna.

According to another aspect of the present invention, there is provided a signal processing method of an RF communication device including: receiving a chaotic carrier generated by inserting a data signal into a chaotic signal including a plurality of frequency components in a predetermined frequency band from an external source; extracting the data signal from the received chaotic carrier; and processing the extracted data signal so as to output the extracted data signal.

According to another aspect of the present invention, there is provided a signal processing method of an RF communication device including: generating a chaotic signal including a plurality of frequency components in a predetermined frequency band; inserting a data signal including information to be transmitted into the chaotic signal to generate a chaotic carrier; and transmitting the chaotic carrier to another external device.

The signal processing method may further include converting an analog data signal into a digital data signal to generate the data signal.

### BRIEF DESCRIPTION OF THE DRAWINGS

The above aspects and features of the present invention will be more apparent by describing certain exemplary embodiments of the present invention with reference to the accompanying drawings, in which:

FIG. 1 is a view illustrating a transmission of data between a portable remote terminal and an RF headset respectively including RF communication systems;

FIG. 2 is a view illustrating a configuration of an RF communication device using a chaotic signal and waveforms at points \textcircled{a} through \textcircled{g} according to an exemplary embodiment of the present invention;

FIG. 3A is a graph showing a chaotic signal generated by a chaotic signal generator shown in FIG. 2 in a frequency domain;

FIG. 3B is a graph showing the chaotic signal shown in FIG. 3A in a time domain;

FIG. 3C is a graph illustrating an enlarged data signal;

FIG. 3D is a graph showing a chaotic carrier modulated from the data signal of FIG. 3C using a chaotic signal in a frequency domain;

FIG. 3E is a graph showing the chaotic carrier of FIG. 3D in a time domain;

FIG. 4A is a block diagram of an RF communication device using a chaotic signal according to an exemplary embodiment of the present invention;

FIG. 4B is a block diagram of an RF communication device using a chaotic signal according to another exemplary embodiment of the present invention; and

FIG. 5 is a flowchart of a process of transmitting and/or receiving data between a portable remote terminal and an RF headset according to an exemplary embodiment of the present invention.

### DETAILED DESCRIPTION OF EXEMPLARY EMBODIMENTS OF THE INVENTION

Certain exemplary embodiments of the present invention will be described in greater detail with reference to the accompanying drawings.

In the following description, same drawing reference numerals are used for the same elements even in different drawings. The matters defined in the description such as a detailed construction and elements are nothing but the ones provided to assist in a comprehensive understanding of the invention. Thus, it is apparent that the present invention can be carried out without those defined matters. Also, well-known functions or constructions are not described in detail since they would obscure the invention in unnecessary detail.

In the present invention, an RF communication system transmitting and/or receiving data using a chaotic signal is applied to each of a main device and a sub-device performing radio communications therebetween. Here, the chaotic signal is a kind of carrier having a plurality of frequency components in a predetermined frequency band and generated directly in a frequency band for transmitting a data signal.

The main device of the RF communication system may be a portable remote terminal, a computer, an electric home appliance, an MP3 player, an RF transceiver installed at a bus stop, or the like, and the sub-device may be a computer peripheral device such as an RF headset, a scanner, a printer, or the like.

According to an exemplary embodiment of the present invention, a main device and a sub-device may be respectively a portable remote terminal and an RF headset. However, an RF communication system-according to the present exemplary embodiment may be applied to all types of devices capable of performing local area radio communications.

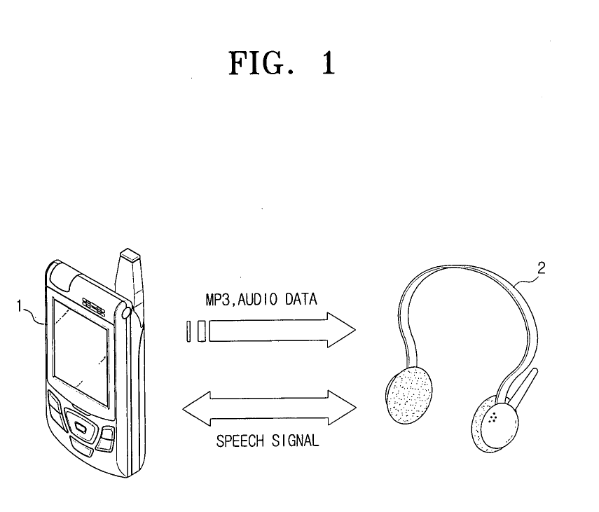

FIG. 1 is a view illustrating a transmission of data between a portable remote terminal and an RF headset respectively including RF communication systems.

A portable remote terminal 1 may communicate with another portable remote terminal or a server, and output a data signal provided from a base station through a screen or a speaker or store the data signal in a memory.

An RF headset 2 transmits and/or receives an RF signal such as MPEG Audio Layer-3 (MP3) data, audio data, a speech signal, or the like with the portable remote terminal 1 through local area radio communications. The local area radio communications between the RF headset 2 and the portable remote terminal 1 may be two-way radio communications or one-way radio communications according to the type of data.

For example, during a telephone call through the portable remote terminal 1, a speech signal arriving at the portable remote terminal 1 is provided to the RF headset 2 to be output to the speaker. Also, sound input through a microphone of the RF headset 2 is modulated to an RF signal, provided to the portable remote terminal 1, and transmitted to the base station. In other words, the speech signal is transmitted and/or received between the portable remote terminal 1 and the RF headset 2, i.e., bi-directionally. Data stored in the portable remote terminal 1 or provided through an RF Internet, for example, audio data compressed into MP3 data, is transmitted from the portable remote terminal 1 toward the RF headset 2, i.e., in one direction.

Both of the portable remote terminal 1 and the RF headset 2 must include an RF communication system for transmitting and/or receiving an RF signal.

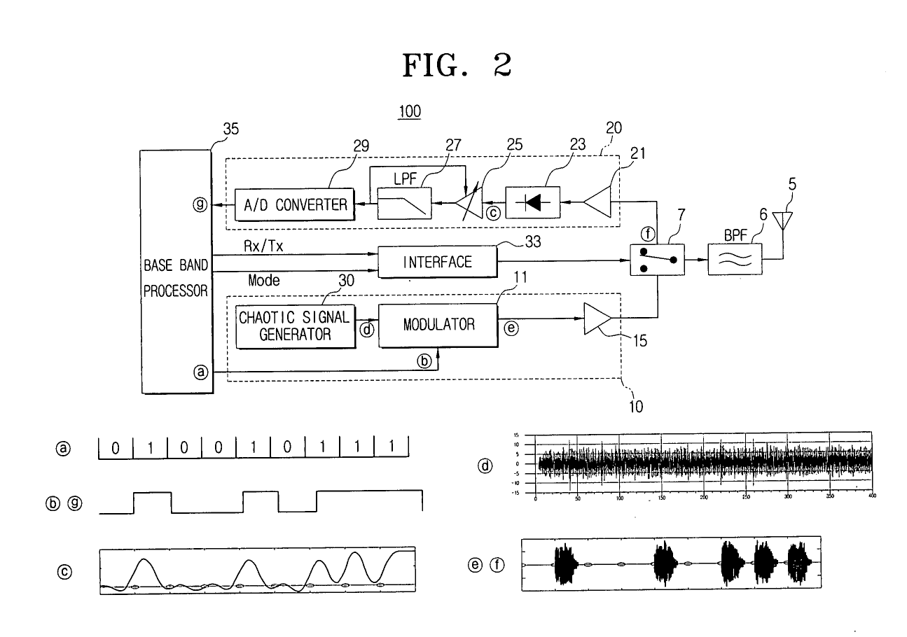

FIG. 2 is a view illustrating a configuration of an RF communication device using a chaotic signal and waveforms at points \textcircled{a} through \textcircled{g} according to an exemplary embodiment of the present invention.

An RF communication device 100 according to the present exemplary embodiment includes a transmitter 10 transmitting a chaotic carrier generated by synthesizing a data signal with a chaotic signal and a receiver 20 receiving the chaotic carrier to extract the data signal. Here, the RF communication device 100 may be a portable remote terminal, a computer, an MP3 player, an RF transceiver, a television (TV), an audio system, a portable multimedia player (PMP), a personal digital assistant (PDA), an RF headset, an RF mouse, a computer peripheral device, or the like.

The RF communication device 100 further includes a switch 7 connecting the transmitter 10 or the receiver 20 to an antenna 5 for transmission and/or reception and a band pass filter (BPF) 6 filtering the chaotic carrier that is transmitted or received. The BPF 6 selectively receives only a signal in a predetermined frequency that may be processed by the RF communication device 100, from the antenna 5 and limits a frequency band of a signal to be transmitted and then transmits the signal to the antenna 5

The RF communication device 100 further includes a base band processor 35 packeting data bits and an interface 33 controlled by the base band processor 35 so as to control an operation of the switch 7. The base band processor 35 packets data bits including information to be transmitted so as to transmit and/or receive an information signal (data bits) with another communication device using the chaotic signal as a carrier, transmits the packeted data bits to the transmitter 10, and converts input packeted data into data bits.

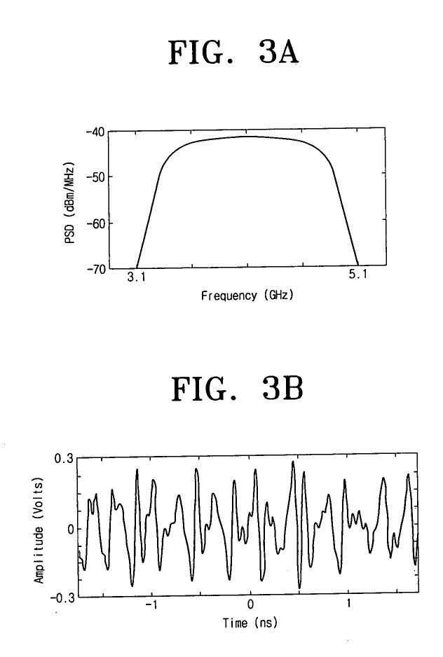

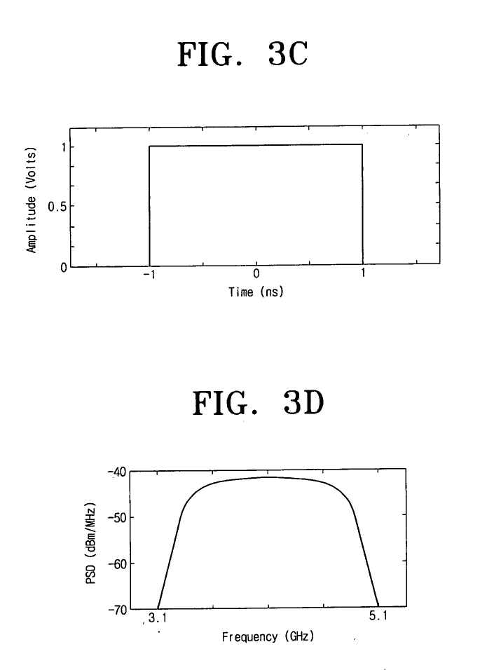

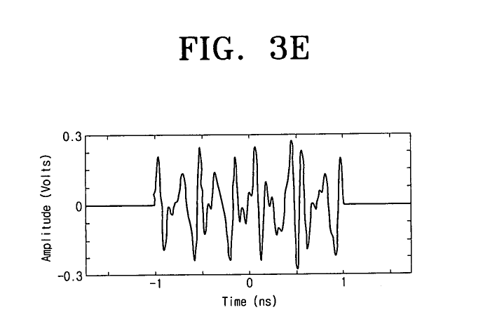

The transmitter 10 includes a chaotic signal generator 30, a modulator 11, and a power amplifier 15. The chaotic signal generator 30 generates a chaotic signal having a plurality of frequency components in a preset predetermined frequency band. The chaotic signal is formed of a plurality of consecutive pulses having non-uniform periods and amplitudes in a time domain as shown in a graph of a point \textcircled{d} of FIG. 2. FIG. 3A is a graph showing the chaotic signal at the point \textcircled{d} in a frequency domain, and FIG. 3B is a graph showing a portion of the chaotic signal at the point \textcircled{d} enlarged in a time domain. As shown in FIG. 3A, the chaotic signal is widely formed in the preset predetermined frequency band. A frequency band of the chaotic signal may vary with a design of the chaotic signal generator 30. Also, as shown in FIG. 3A, the chaotic signal may be widely formed in a frequency band between 3.1 GHz and 5.1 GHz as a UWB band.

The modulator 11 synthesizes the chaotic signal generated by the chaotic signal generator 30 with the data signal output from the base band processor 35 to generate the chaotic carrier. Here, binary data bits formed of “0” and “1” as shown in a graph of a point \textcircled{a} are provided as pulse forms shown in a graph of a point \textcircled{b} to the modulator 11. The data signal having the pulse forms provided to the modulator 11 may be shown in a graph of FIG. 3C. If the data signal is synthesized with the chaotic signal, a chaotic carrier in which a chaotic signal exists only in a section of the data signal having information as shown in a graph of a point \textcircled{e}. FIG. 3D is a graph showing the chaotic carrier at the point \textcircled{e} in a frequency domain, and FIG. 3E is a graph showing the chaotic carrier enlarged in a time domain. As shown in FIG. 3D, after the chaotic carrier is completely modulated, a frequency band of the chaotic carrier is almost equal to the frequency band of the chaotic signal shown in FIG. 3A.

When the transmitter 10 operates, the interface 33 controls the switch 7 to connect the transmitter 10 to the antenna 5, and the chaotic carrier generated by the transmitter 10 is filtered by the BPF 6 and then transmitted to another external communication device through the antenna 5.

The receiver 20 includes a low noise amplifier (LNA) 21, a detector 23, an automatic gain control (AGC) amplifier 25, a low pass filter (LPF) 27, and an analog-to-digital converter (ADC) 29 and processes an RF signal received from an external source. In the present embodiment of the invention, the data signal may be a speech signal, a video signal, or a control signal through which a transmitter terminal controls a receiver terminal and is not limited to a specific type of data signal.

The LNA 21 of the receiver 20 amplifies the chaotic carrier received through the antenna 5 and then transmits the chaotic carrier to the detector 23. The detector 23 senses an envelope of the chaotic carrier and extracts a data signal. The detector 23 may be a diode, and the chaotic carrier having passed through the detector 23, forms a signal having waveforms as shown in a graph of a point \textcircled{c}. The AGC amplifier 25 is an amplifier capable of increasing and/or decreasing an amplification factor and amplifies the signal extracted by the detector 23 to a predetermined level. The LPF 27 filters the amplified waveforms so that the ADC 29 converts the amplified waveform into a digital signal. The ADC 29 converts a data stream into a digital signal and extracts a data signal having a pulse form as shown in FIG. 3C.

The RF communication device 100 shown in FIG. 2 includes the transmitter 10 and the receiver 20 but may include only the transmitter 10 or the receiver 20. For example, an RF earphone may include only the receiver 20, and a portable remote terminal or an RF headset may include both the transmitter 10 and the receiver 20.

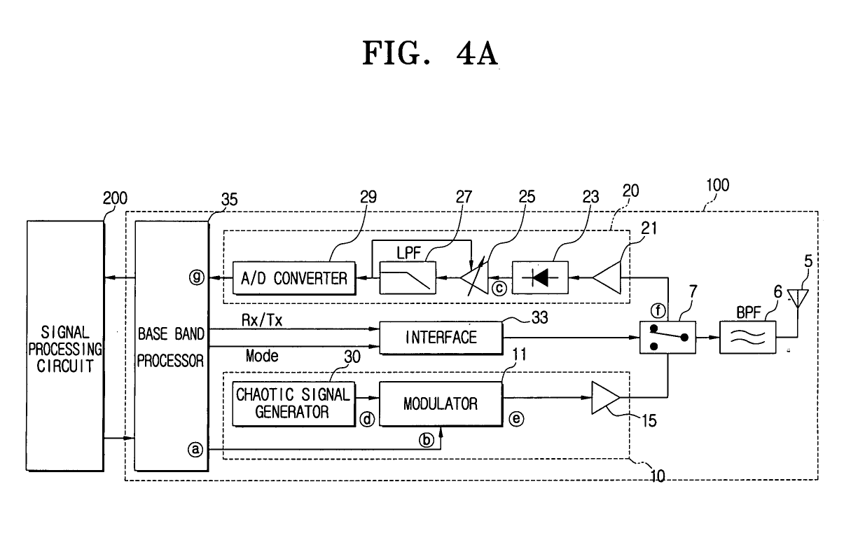

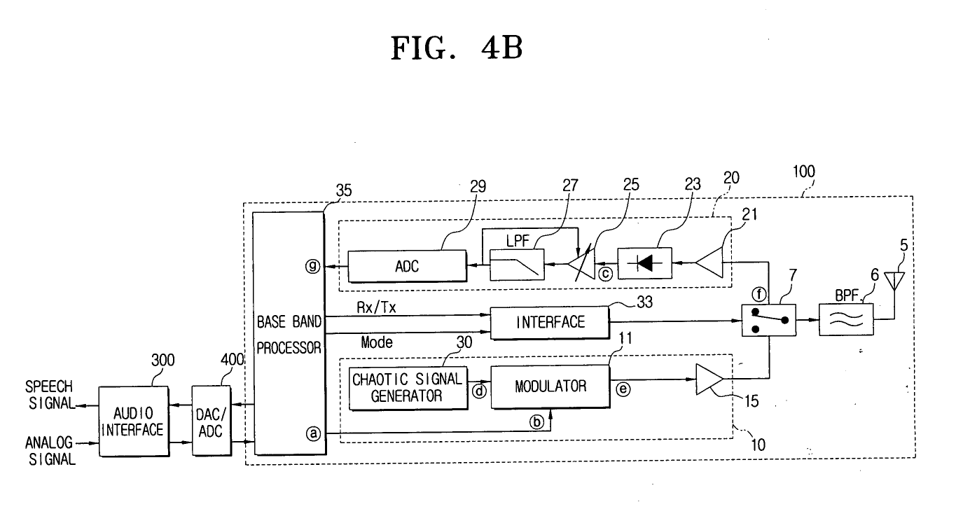

FIG. 4A is a block diagram of an RF communication device using a chaotic signal according to an exemplary embodiment of the present invention, and FIG. 4B is a block diagram of an RF communication device using a chaotic signal according to another exemplary embodiment of the present invention.

An RF communication device shown in FIG. 4A may be the RF communication device 100 of FIG. 2 applied to a portable remote terminal. Thus, a transmitter 10 and a receiver 20 shown in FIG. 4A have the same structures as the transmitter 10 and the receiver 20 shown in FIG. 2, respectively. In other words, a core part 100 of an RF communication device using a chaotic signal is applied to a portable remote terminal. The core part 100 of the RF communication device is connected to a signal processing circuit 200 processing a code division multiple access (CDMA) signal for mobile communication, and the signal processing circuit 200 provides a base band processor 35 with data bits including information to be transmitted. The signal processing circuit 200 may be a mobile station modem that is a central processing unit (CPU) of a portable remote terminal.

An RF communication device shown in FIG. 4B may be the core part 100 of the RF communication device of FIG. 2 applied to an audio device and thus further includes an audio interface 300 and an ADC 400. The audio interface 300 receives an analog signal through a microphone from an external source or from an audio device (not shown) installed and operating together with the core part 100 of the RF communication device. The analog signal input through the audio interface 300 is converted into a digital data signal by the ADC 400 and then provided to the base band processor 35. Also, a signal processing operation of a rear end of the base band processor 35 is as described in the previous exemplary embodiment. When the digital data signal provided through the ADC 400 from the receiver 20 is converted into an analog data signal, the audio interface 300 outputs a speech signal corresponding to the analog data signal.

Although not shown, the signal processing circuit 200 shown in FIG. 4A and the audio interface 300 and the ADC 400 shown in FIG. 4B may all be installed in an RF communication device according to exemplary embodiments of the present invention. The signal processing circuit 200, the audio interface 300, and the ADC 400 may be integrated into one chip along with the core part 100 of the RF communication device

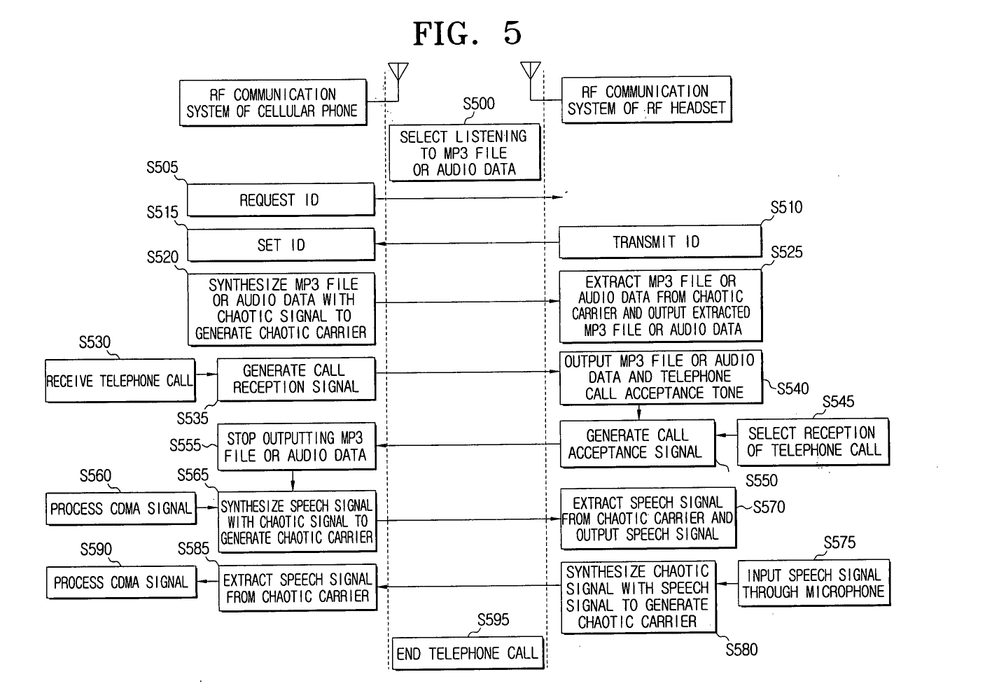

A process of transmitting and/or receiving data between the portable remote terminal 1 and the RF headset 2 including RF communication systems having the above-described structure will now be described with reference to FIG. 5.

If a user desires to listen to an MP3 file or audio data stored in the portable remote terminal 1 or downloaded through an access to a wireless Internet, the user manipulates a button of the portable remote terminal 1 or the RF headset 2 to select an output of the audio data in operation S500. In operation S505, the portable remote terminal 1 transmits a signal for requesting an identification (ID) to the RF headset 2. In operation S510, the RF headset 2 transmits the ID to the portable remote terminal 1. If the portable remote terminal 1 determines that the ID provided from the RF headset 2 is not equal to an ID stored in the portable remote terminal 1, the portable remote terminal 1 displays a message announcing an impossibility of transmission of the MP3 file or the audio data.

If the ID provided from the RF headset 2 is equal to the ID stored in the portable remote terminal 1, the portable remote terminal 1 sets the corresponding ID and processes the MP3 file or the audio data as an RF signal in operation S515. Here, the transmitter 10 of the portable remote terminal 1 provides an MP3 file or audio data having a data bit format stored in a random memory to the base band processor 35, and the base band processor 35 packets the MP3 file or the audio data to generate a data signal. In operation S520, the chaotic signal generator 30 generates a chaotic signal, and the modulator 11 synthesizes the chaotic signal with the data signal to generate a chaotic carrier.

If transmitter 10 operates, the interface 33 operates the switch 7 to connect the transmitter 10 to the antenna 5. The chaotic carrier of the MP3 file or the audio data is then filtered by the BPF 6 and then transmitted to the RF headset 2 through the antenna 5.

If the chaotic carrier is received, the interface 33 of the RF headset 2 operates the switch 7 to connect the receiver 20 to the antenna 5. The chaotic carrier received through the antenna 5 of the RF headset 2 is amplified by the LNA 21, and the detector 23 senses and extracts the data signal from the chaotic carrier. The extracted data signal is amplified and filtered by the AGC amplifier 25 and the LPF 27, respectively, and then provided to the ADC 29. The ADC 29 converts the data signal into a digital signal, and the base band processor 35 de-packets the data signal and provides the de-packeted data signal to the audio interface 300. In operation S525, the audio interface 300 converts data bits into an analog signal and outputs the analog signal through a speaker.

If a telephone call is received from an external source during the reception of the MP3 file or the audio data in operation S530, the portable remote terminal 1 generates a call reception signal including information as to the reception of the telephone call and transmits the call reception signal to the RF headset 2 in operation S535. In operation S540, the RF headset 2 outputs a telephone call acceptance tone together with the MP3 file or the audio data. If the user selects the telephone call using the button of the RF headset 2 in operation S545, a call acceptance signal for announcing the selection of the telephone call is generated and transmitted to the portable remote terminal 1 in operation S550.

The transmitter 10 of the portable remote terminal (a cellular phone) 1 stops transmitting the MP3 file or the audio data in operation S555 and provides a CDMA signal processed by the signal processing circuit 50 to the base band processor 35 in operation S560. Here, the CDMA signal is packeted by the base band processor 35 and then provided to the modulator 11. In operation S565, the modulator 11 synthesizes the packeted CDMA signal with a chaotic signal to generate a chaotic carrier.

The chaotic carrier is transmitted to the RF headset 2, and the interface 33 of the RF headset 2 switches on the switch 7 to connect the receiver 20 to the antenna 5. The receiver 20 extracts a data signal from the chaotic carrier, and the base band processor 35 de-packets the data signal and provides the de-packeted data signal to the audio interface 300. In operation S570, the audio interface 300 converts data bits into an analog signal and output the analog signal through the speaker.

In operation 575, the speech signal of a user is input through a microphone. The speech signal is input to the audio interface 300, converted into a digital signal by the ADC 400, and provided to the base band processor 35, and the base band processor 35 packets data bits. In operation S580, the modulator 11 synthesizes the chaotic signal with the data signal to generate a chaotic carrier and transmits the chaotic carrier to the portable remote terminal 1. In operation S585, the chaotic carrier input to the receiver 20 of the portable remote terminal 1 is converted into data bits and then provided to the signal processing circuit 200. In operation S590, the signal processing circuit 200 converts the data bits into a CDMA signal.

Operations S560 through S590 are repeated until the telephone call is ended.

As described above, in RF communication devices using a chaotic signal and a method thereof according to the exemplary embodiment of the present invention, a chaotic signal that is a UWB high frequency signal can be used as a chaotic carrier. Thus, each of a transmitter and a receiver does not additionally require VCOs, PLLs, up-mixers, down-mixers, and so on. Thus, power consumption of the RF communication devices can be considerably reduced, and sizes of the RF communication devices can be reduced.

Accordingly, low power, compact, and light local area RF communication devices can be realized.

The foregoing embodiments and advantages are merely exemplary and are not to be construed as limiting the present invention. The present teaching can be readily applied to other types of apparatuses. Also, the description of the exemplary embodiments of the present invention is intended to be illustrative, and not to limit the scope of the claims, and many alternatives, modifications, and variations will be apparent to those skilled in the art.

What is claimed is:

1. An RF (radio frequency) communication system comprising:
a transmitter terminal which generates a chaotic signal comprising a plurality of frequency components in a predetermined frequency band and transmits a chaotic carrier generated by synthesizing a data signal with the chaotic signal to a receiver terminal; and
the receiver terminal extracts the data signal from the chaotic carrier received from the transmitter terminal via a wireless network.

2. The RF communication system of claim 1, wherein the transmitter terminal comprises:
a chaotic signal generator which generates the chaotic signal;
a modulator which synthesizes the data signal with the chaotic signal to generate the chaotic carrier; and
an antenna which transmits the chaotic carrier generated by the modulator.

3. The RF communication system of claim 1, wherein the receiver terminal comprises a detector which senses the chaotic carrier and extracts the data signal.

4. The RF communication system of claim 1, wherein the transmitter terminal comprises a signal processing circuit which converts the data signal to a base band and outputs a digital signal.

5. The RF communication system of claim 4, wherein the signal processing circuit is a mobile station modem of a portable remote terminal.

6. The RF communication system of claim 1, wherein the transmitter terminal comprises an audio interface which receives an analog data signal from an external source.

7. The RF communication system of claim 6, wherein the transmitter terminal further comprises an ADC (analog-to-digital converter) which converts an analog data signal output from the audio interface into a digital data signal.

8. The RF communication system of claim 1, wherein the receiver terminal comprises a DAC which converts the extracted digital data signal into an analog data signal.

9. The RF communication system of claim 8, wherein the receiver terminal further comprises an audio interface which outputs the analog data signal.

10. The RF communication system of claim 1, wherein the transmitter terminal is one of a portable remote terminal, a computer, an MP3 (MPEG Audio Layer-3) player, an RF transceiver, a TV (television), an audio system, a PMP (portable multimedia player), a PDA (personal digital assistant), an RF headset, an RF mouse, and a computer peripheral device.

11. The RF communication system of claim 1, wherein the receiver terminal is one of a portable remote terminal, a computer, an MP3 player, an RF transceiver, a TV, an audio device, a PMP, a PDA, an RF headset, an RF mouse, a computer peripheral device, and a speaker.

12. An RF communication device comprising:
a chaotic signal generator which generates a chaotic signal comprising a plurality of frequency components in a predetermined frequency band, and which transmits a chaotic carrier generated by synthesizing a data signal comprising information to be transmitted with the chaotic signal, to a receiver terminal.

13. The RF communication device of claim 12, further comprising
a modulator which synthesizes the data signal with the chaotic signal to generate a chaotic carrier; and an antenna which transmits the chaotic carrier generated by the modulator.

14. The RF communication device of claim 12, further comprising a signal processing circuit which converts the data signal to a base band and outputs a digital signal.

15. The RF communication device of claim 14, wherein the signal processing circuit is a mobile station modem of a portable remote terminal.

16. The RF communication device of claim 12, further comprising an audio interface which receives an analog data signal.

17. The RF communication device of claim 16, further comprising an ADC which converts an analog data signal output from the audio interface into a digital data signal.

18. The RF communication device of claim 12, wherein the RF communication device is at least one of a portable remote terminal, a computer, an MP3 player, an RF transceiver, a TV, an audio device, a PMP, a PDA, an RF headset, an RF mouse, and a computer peripheral device.

19. An RF communication device comprising:
an antenna which receives a chaotic carrier generated by inserting a data signal into a chaotic signal comprising a plurality of frequency components in a predetermined frequency band via a wireless network, extracts the data signal from the chaotic carrier, and processes the data signal.

20. The RF communication device of claim 19, further comprising a detector which senses the chaotic carrier and extracts the data signal.

21. The RF communication device of claim 20, wherein the detector is a diode which detects an envelope of the data signal from the chaotic carrier.

22. The RF communication device of claim 19, further comprising:
a DAC which converts he extracted digital data signal into an analog data signal; and an audio interface which outputs the analog data signal.

23. The RF communication device of claim 19, wherein the RF communication device is one of a portable remote terminal, a computer, an MP3 player, an RF transceiver, a TV, an audio device, a PMP, a PDA, an RF headset, an RF mouse, a computer peripheral device, and a speaker.

24. An RF communication device comprising:
a transmitter which generates a chaotic signal comprising a plurality of frequency components in a predetermined frequency band and transmits a chaotic carrier generated by inserting a data signal comprising information to be transmitted into the chaotic signal to a receiver;
wherein the receiver extracts the data signal from the chaotic carrier received through an antenna and processes the extracted data signal.

25. The RF communication device of claim 24, further comprising:
a switch which connects one of the transmitter and the receiver to the antenna; and
a BPF (band pass filter) which filters the chaotic carrier transmitted or received through the antenna.

26. The RF communication device of claim 24, further comprising a duplexer which connects one of the transmitter and the receiver to the antenna and filters the chaotic carrier transmitted or received through the antenna.

27. The RF communication device of claim 24, wherein the transmitter comprises:
a chaotic signal generator which generates the chaotic signal; and
a modulator which synthesizes the data signal with the chaotic signal to generate the chaotic carrier;
wherein the chaotic carrier generated by the modulator is transmitted by the antenna.

28. The RF communication device of claim 24, wherein the receiver comprises a detector which senses an envelope of the received chaotic carrier and extracts the data signal.

29. The RF communication device of claim 24, wherein the transmitter further comprises a signal processing circuit which converts the data signal to a base band and outputs a digital signal.

30. The RF communication device of claim 29, wherein the signal processing circuit is a mobile station modem of a portable remote terminal.

31. The RF communication device of claim 24, wherein the transmitter further comprises an audio interface which receives an analog data signal from an external source.

32. The RF communication device of claim 31, wherein the transmitter further comprises an ADC which converts the analog data signal output from the audio interface into a digital data signal.

33. The RF communication device of claim 24, wherein the receiver comprises:
a DAC which converts the extracted digital data signal into an analog data signal; and
an audio interface which outputs the analog data signal.

34. The RF communication device of claim 24, wherein the RF communication device is at least one of a portable remote terminal, a computer, an MP3 player, an RF transceiver, a TV, an audio device, a PMP, a PDA, an RF headset, an RF mouse, and a computer peripheral device.

35. A signal processing method of an RF communication device comprising:
receiving a chaotic carrier generated by inserting a data signal into a chaotic signal comprising a plurality of frequency components in a predetermined frequency band from an external source;
extracting the data signal from the received chaotic carrier; and
processing the extracted data signal so as to output the extracted data signal.

36. A signal processing method of an RF communication device comprising:
generating a chaotic signal comprising a plurality of frequency components in a predetermined frequency band;
inserting a data signal comprising information to be transmitted into the chaotic signal to generate a chaotic carrier; and
transmitting the chaotic carrier to an external device.

37. The signal processing method of claim 36, further comprising converting an analog data signal into a digital data signal to generate the data signal.
